(19) United States
(12) Patent Application Publication
(10) Pub. No.: US 2025/0166183 A1
(43) Pub. Date: May 22, 2025

(54) WOUND IMAGE GATHERING AND CLARIFICATION

(71) Applicant: Worcester Polytechnic Institute, Worcester, MA (US)

(72) Inventors: Emmanuel Agu, Worcester, MA (US); Akshay Iyer, Worcester, MA (US); Raghav Nagpal, Worcester, MA (US); Apiwat Ditthapron, Worcester, MA (US); Ziyang Liu, Worcester, MA (US); Reza Sadaarti, Worcester, MA (US); Bengisu Tulu, Worcester, MA (US); Diane Strong, Worcester, MA (US); Peder Pedersen, Worcester, MA (US); Raymond Dunn, Worcester, MA (US); Clifford Lindsay, Worcester, MA (US)

(21) Appl. No.: 18/953,632

(22) Filed: Nov. 20, 2024

Related U.S. Application Data
(60) Provisional application No. 63/601,114, filed on Nov. 20, 2023.
(60) Provisional application No. 63/601,117, filed on Nov. 20, 2023.

(51) Int. Cl. G06T 7/00; G06T 3/40; G06T 5/73; G06T 5/90; G16H 30/40; G16H 50/30

(52) U.S. Cl. CPC G06T 7/0012; G06T 3/40; G06T 5/73; G06T 5/90; G16H 30/40; G16H 50/30; G06T 2207/20081; G06T 2207/30088

(57) ABSTRACT

A wound care treatment platform and application employs a mobile device and application (“app”) for on-site capture and gathering of wound images from a patient. The mobile device is in wireless communication with a database including health records and data and trained models of wound image classification. Based on a patient image of a wound under care, the image is analyzed for features indicative of wound health and healing progress. The mobile device invokes a plurality of models for providing an accurate and consistent assessment and treatment recommendation, including evaluating the sufficiency of the patient image gathered by the mobile device, normalizing the patient image for adverse or irregular lighting, common in patient dwellings, adjusting for a distance and angle at which the caretaker obtained the image, computing a comprehensive score of wound healing, and rendering an evaluation for referral or continuance of current outpatient care.

### RELATED APPLICATIONS

This patent application claims the benefit under 35 U.S.C. § 119 (e) of U.S. Provisional Patent App. No. 63/601,114, filed Nov. 20, 2023, entitled “WOUND ASSESSMENT AND CLASSIFICATION,” and U.S. Provisional Patent App. No. 63/601,117, filed Nov. 20, 2023 entitled “WOUND IMAGE GATHERING AND CLARIFICATION,” both incorporated herein by reference in entirety.

### STATEMENT OF FEDERALLY SPONSORED RESEARCH AND DEVELOPMENT

This invention was made, at least in part, with U.S. Government support under Contract No. NIH/NIBIB 1R01EB025801-01, awarded by the National Institute for Health (NIH). The Government has certain rights in the invention.

### BACKGROUND

Wound care is a significant aspect of medical treatment, and one which is often delegated to outpatient services that travel to patient's homes. Since a wound represents a breach of the epidermal surface, which is a primary barrier to infections and disease, proper wound care ensures proper healing and guards against exacerbating conditions, which may lead to a necessity of inpatient care or worse. Such outpatient services employ nurses and medical technicians to visit the patient at home, examine the wound, evaluate healing progress, and consider changes to treatment, such as referral to an inpatient setting or specialist.

### SUMMARY

A wound care treatment platform and application employs a mobile device and application (“app”) for on-site capture and gathering of wound images from a patient. The mobile device is in wireless communication with a database including health records and data, and trained models of wound image classifications. Based on a patient image of a wound under care, the image is analyzed for features indicative of wound health and healing progress. The mobile device invokes a plurality of models for providing an accurate and consistent assessment and treatment recommendation, including evaluating the sufficiency of the patient image gathered by the mobile device, normalizing the patient image for adverse or irregular lighting, common in patient dwellings, adjusting for a distance and angle at which the caretaker obtained the image, computing a comprehensive score of wound healing, and rendering an evaluation for referral or continuance of current outpatient care.

Configurations herein are based, in part, on the observation that mobile or remote medical services, typically in-home visits and care by a travelling caretaker, are a popular alternative to inpatient care. Hospital on-site resources are saved, and patients need not incur travel overhead, while the periodic home visits can mitigate exacerbation or unexpected degradation of a patient healing process. Unfortunately, conventional approaches to in-home visits suffer from the shortcoming that caretaker experience and capabilities can vary, and subjective assessment of possibly worsening conditions may escape diagnosis or identification. Particularly with in-home wound care, visible indications of degraded or insufficiently healed wounds may not be recognized, and the need for timely referral missed.

Accordingly, configurations herein substantially overcome the shortcomings of conventional in-home wound care by providing a mobile app and supporting network service that ensures gathering of a sufficiently descriptive patient wound image, normalizes and recreates the patient image for environmental anomalies, and analyzes and scores the patient image for computing a scored assessment of wound health and a recommendation for future care.

In further detail, configurations described herein provide method of gathering and classifying wound images by receiving a patient image from a personal device, such that the patient image contains an image of a wound under care, where the image of the wound under care was gathered with an ambient lighting, angle and distance of the personal device relative to the wound under care. A smartphone app, in conjunction with a server app, evaluates a sufficiency of a patient image for analysis, the patient image depicting a wound under care, and normalizing, if the patient image is sufficient for analysis, a shading of the patient image for comparison with other wound images, the shading based on the ambient lighting. The application then reconstructs the patient image for accommodating variations in the angle and the distance, and analyzing the reconstructed image for rendering a recommendation including the wound score and the additional care.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a system context diagram including a mobile device and smartphone app for capturing a visual patient image of a wound under care, and evaluating the patient image using a wireless link or connection;

FIG. 2 is a process flow of the wound care and analysis application of FIG. 1;

FIG. 3 shows the average relative accuracy and percentage of rejected wound image;

FIG. 4 shows results of lighting correction by the lighting model;

FIG. 5 shows a flowchart of the reconstruction and rectification process;

FIG. 6 shows the transforms in the reconstruction of FIG. 5;

FIGS. 7A-7B show the learning architecture for accessing 8 PWAT sub-scores for the wound healing model;

FIG. 7C shows the deployment or production use of the wound healing model;

FIG. 8 shows a Multimodal Classifier Pipeline implementing fusion of expertise from Natural Language Processing (NLP) and Convolutional Neural Networks (CNN) for robust decision-making; and

FIG. 9 shows intermediate fusion of multimodal features as in FIG. 8.

### DETAILED DESCRIPTION

The description below presents an example of a mobile application (“app”), database and classification models of a system for patient in-home wound assessment and decision support for future care. Other database and classification models may be employed in the manner described herein, and various processing and load sharing/balancing approaches between the mobile device resident application and classification model processing for wound evaluation.

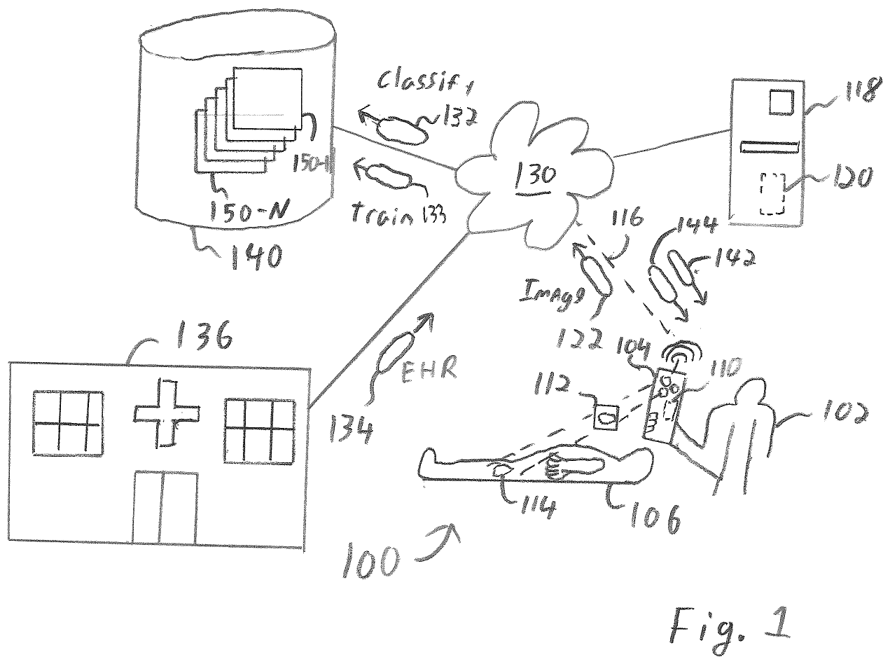

FIG. 1 is a system context diagram including a mobile device and smartphone app for capturing a visual patient image of a wound under care, and evaluation of the patient image using a wireless link. Referring to FIG. 1, in a healthcare environment 100, a caretaker 102 or healthcare worker invokes a cellphone app 110 on their mobile device 104 (cellphone, smartphone, tablet or other portable processing and telecommunications device). The mobile device 104 takes and receives an image 112 of a wound 114 of a patient 106, which the app 110 receives. A wireless interface 116 sends a message 122 including the image 112 to a remote server 118 via a public access network 130 or intranet. The server 118 launches and executes a server application 120 to invoke a database 140 with a plurality of models 150-1 . . . 150-N (150 generally). A classification request 132 from the server 118 commences analysis and classification of the image 112 using the models 150 in the database 140. The models 150 are trained on information 133 including previous data, images, and patient diagnoses and classification labels from electronic health records 134 from a hospital 136 or health records repository (anonymized for privacy concerns). Based on the analysis, the server 118 sends a wound score 142 and additional care 144 needed for the patient.

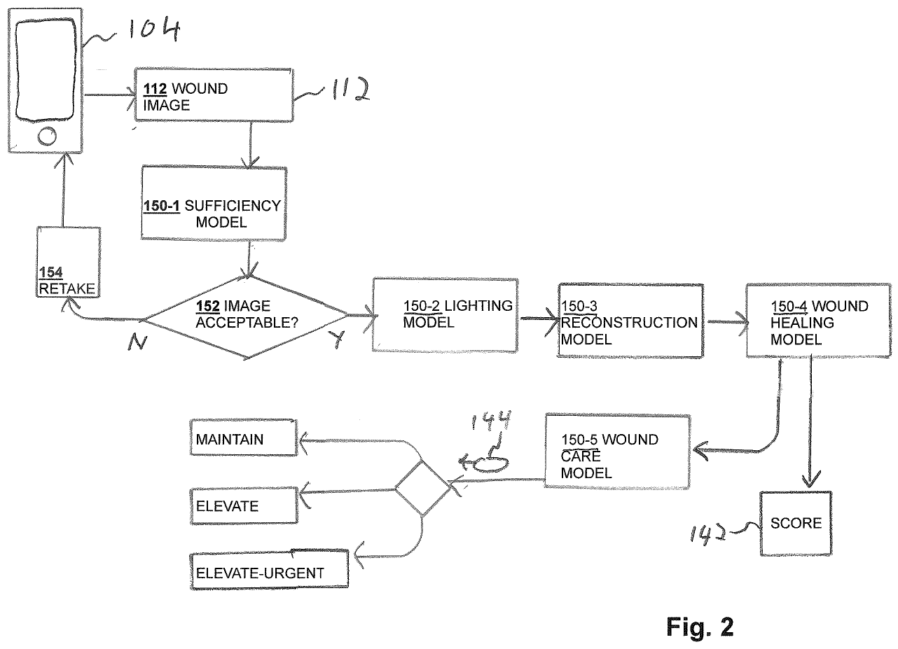

FIG. 2 is a process flow of the wound care and analysis application of FIG. 1. The database 140 includes several models for classification using machine learning (ML) and artificial intelligence (AI) classification and probabilistic approaches, discussed further below. The result is a decision support and wound score output for allowing uniform and consistent recommendations and guidance to in-home caretakers pursuing patient wound care follow-up. Referring to FIGS. 1 and 2, in a particular example, the smartphone app 110 and server app 120 perform a method for remote monitoring of wound healing and care by receiving the patient image 112 from a personal device 104, such that the image 112 contains an image of a wound under care 114, and based on mobile device capabilities in conjunction with an ambient lighting, angle and distance of the personal device relative 104 to the wound under care 114. The transmitted message 122 with the patient image 112 is received by a sufficiency model 150-1 for evaluating a sufficiency of a patient image for analysis, where the patient image 112 depicts the wound under care 114. The sufficiency model 150-1 evaluates the patient image, and a check is performed, at step 152. If the image is found insufficient for further processing, such as due to insufficient lighting, angle, movement/jitter, transmission encoding error, etc., the mobile device 104 receives a message to retake the patient image 112, at step 154.

If the patient image 112 is sufficient for analysis, the patient image is passed to a lighting model 150-2 for normalizing a shading of the patient image 112 for comparison with other wound images, where the shading is generally based on the ambient lighting, shadows and other conditions at the patient location. An excess of dark or light pixels, or misleading shading due to extreme angles of ambient light, are normalized and corrected, so that comparison of the patient image 112 with images in other models 150 can be performed.

The normalized patient image 112 is then reconstructed for accommodating variations in the angle and the distance, by invoking a reconstruction model 150-3. The reconstruction model 150-3 performs a reconstruction such that multiple patient images 112 appear at a common fixed distance and orthogonal orientation to the wound under cate 114. In other words, it accommodates variations in angle and distance from the wound 114 that may vary among different caretakers 102 and devices 104.

A wound healing model 150-4 generates a wound score 142 based on the reconstructed patient image 112, where the score is indicative of a healing progress of the wound under care 114, discussed further below. The wound care model 150-5 computes, based on a comparison of images of other wounds with the patient image 112, whether additional care is needed for the wound under care 114. The mobile device 104 receives and renders a recommendation including the wound score 142 and the additional care 144 recommendation. While the wound score 142 and additional care 144 may be rendered together or simultaneously, each is computed independently by the respective database and model 150. While a poor wound score might tend to correlate with a need for immediate care, a wound evaluated shortly after surgery may indicate a score in need of additional healing although the condition is as expected for the timeframe. Further, the sufficiency model 150-1, lighting model 150-2, and reconstruction model 150-3 are shown in series, the need not be applied in succession, and each of the models 150 are independently applicable to classifying a patient image 112.

Evaluation using sufficiency model 150-1 for ensuring patient image is sufficient for analysis:

The sufficiency model 150-1 allows image quality assessment on the smartphone 104, enabling low-quality wound images 112 to be rejected immediately after image capture so that the picture taker can be prompted to recapture the image. This preferable commences the smartphone-based AI-driven wound assessment app 110, which addresses a need for IT products that facilitate fast, accurate wound assessment and treatment recommendation in patients' homes (outside the clinic/hospital/office).

Low quality wound images that have luminance, blur, and image compression artifacts can reduce the performance of deep learning models that assess wounds from smartphone images. To improve the performance of the downstream models 150-2 . . . 150-5 for analyzing smartphone wound images, instant image quality assessment on the smartphone 104 allows low-quality wound images to be rejected immediately after image capture and the device 104 may prompt the nurse or patient to recapture the image.

The sufficiency model 150-1 or database evaluates a sufficiency of a patient image depicting a wound under care for analysis. If the patient image 112 is insufficient for analysis, at step 152, the app 110/120 discards the patient image 112, and renders an indication, via the personal device, that the image is insufficient for analysis and indicates a need for a subsequent image gathering. By immediately evaluating whether the image 112 is usable, the caretaker 102 can simply retake the image, rather than concluding that the image is unusable after the caretaker 102 leaves the patient residence. In the example configuration, the application 110 computes an index for each of brightness, compression, and blurriness of the patient image, and computes an image assessment based on a comparison of the computed indices. The application then rejects the patient image if the image assessment does not meet a sufficiency threshold. As a matter of load balancing, the image sufficiency test at step 152 may occur on the server 118, as well, depending on the capabilities of the mobile device app 110. Configurations herein train a model based on stored images of previously classified wounds, and compare the patient image 112 to the stored images of the model 150-1. This allows computation of the indices of brightness, compression, and blurriness based on the comparison.

Most problems are associated with light conditions are caused by 1) poor lighting, such as under-exposure/overexposure images, shadow areas, and low/high saturation, or 2) an improper camera setup that attempts to compensate for poor lighting, such as noise from using a high ISO value and blurry images from long exposures. One study specifically investigated the problem of blurred images as it was found to be the most prevalent (58%) in crowdsourced images taken using smartphones, reducing the performance of object recognition. Also considered are luminance and image compression which were indicated to have a negative impact, but not as statistically significant as blurry images. Fast and effective quality indices to measure the blur, luminance, and compression of a wound image are considered to reject an image that may lead to inaccurate wound assessment results. The blur index is computed using an image gradient approach and meta information is used to compute luminance and image compression indexes. As depicted in FIG. 1, images 112 that fail quality assessment tests should be rejected by the system, and require the nurse or patient to retake an image in better conditions. This framework improves confidence in the wound assessment and saves computational resources by not analyzing bad images.

In contrast to some approaches, the disclosed approach uses the meta information of an image as a measure of its brightness and compression rate, as well as the gradient magnitude ranking to detect blur, allowing instant quality assessment of wound image on smartphone.

Brightness: The brightness of the image is analyzed in the CIELAB color space. The index I_{Br} is the average value of the L channel across all image pixels P. The nominal range of the L channel is 0 to 100. The index was then normalized to be between 0 and 1 where index of 0 denotes L value of 50 and index of 1 denotes L value of 0 or 100, as in:

$${I}_{Br} = \frac{2}{100 P} {\sum }_{i = 1}^{P} \vert {L}_{i} - 50 \vert$$

Image compression: The disclosed approach considered both a JPEG compression rate and image resolution when analyzing image compression. One approach evaluates image compression in numeric units ranging from 1 to 100 (high compression to low compression); we normalized the index to range from 0 to 1. A typical analysis, operates on images measuring 224 by 224 pixels. We rated images with dimensions greater than 224 as 0 (highest quality) and those with dimensions less than 224 using:

$${I}_{C} = ( w - 20 ) / 204 * ( h - 20 ) / 204$$

where w and h are the width and height of the image. A 20×20 resolution image is too small for DNN. Therefore, we regarded 20 as the minimum acceptable image size.

Blurriness: A sorted coefficient of high frequency is an effective indicator of blur pixels. A blur index encompasses the result from analyzing the sorted coefficient using a high-frequency multiscale fusion and sort transform of gradient magnitudes, which identifies the blur area of an image as masking. Under the constraints of on-device computation, the values of the sorted high-frequency coefficient computed from the entire image using discrete cosine transform (DCT) were used in the disclosed approach without transforming back to full masking. The coefficient is then normalized to fall within the range of 0 to 1 as blur index I_{Bl}.

Table I summarizes the indexes to reject an inadequate wound image, where threshold T is determined by analyzing the index values of the wound image, and its value is discussed in the results section. The bad wound image rejection prompts the nurse to retake an image, if necessary, spontaneously after the image is captured on smartphones.

TABLE I
Bad wound image rejection logic

```
  Input: Wound image x with W × H pixels and Rejection threshold T
  Output: Binary decision to accept or reject x I_{Br} ← 0 // Brightness index foreach Pixel
p in x do
    {L, A, B} ← RGBtoLAB(p)
    I_{Br} ← I_{Br} + |L − 50|
  I_{Br} ← I_{Br}/W/H/50
  // Image Compression index
  I_{C} ← (W − 20)/204 * (H − 20)/204
  C ← [ ] // Blur index
  Split image x into 7 by 7-pixel blocks K
  foreach k in K do
    k\hat{ } ← abs(DCT 2(k))
    k\hat{ }H ← sort(k\hat{ }[i, j]) where i + j ≥ 7
    Append k\hat{ }H to C
  I_{Bl} ← (Average(C) − min(C))/(max(C) − min(C))
  if (I_{Br} + I_{C} + I_{Bl})/3 ≤ T then
    Return Accept
  else
    Return Reject
```

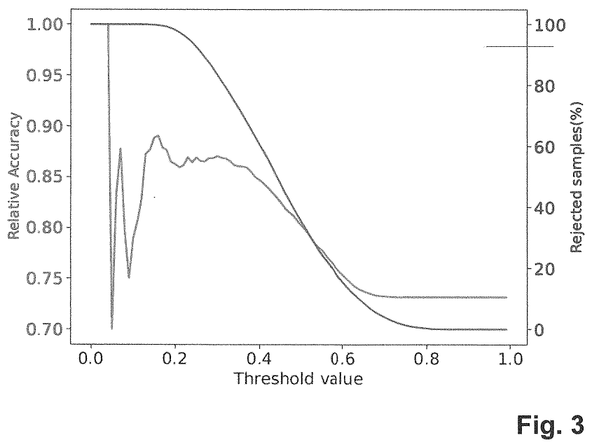

Based on the above, a carefully selected threshold improves the results of deep learning wound assessment by rejecting wound images of insufficient image quality, or above a threshold value. When the average quality index is greater than the threshold, an image is rejected and the nurse or patient is prompted to retake the wound photo with a better set up. The trade-off between relative precision and image rejection is shown in FIG. 3. FIG. 3 shows the average relative accuracy and percentage of rejected wound image. The proposed index increases overall accuracy from 73% to 87.0% by rejecting poor images. However, 20-60% of augmented images must be rejected for the accuracy to begin to improve. Rejecting 50% of augmented images yields an accuracy of 78%. A threshold value around 0.5-0.6 is optimal as performance increases are greatest in this range with a decline after 0.6.

### Normalization of Lighting Conditions of Patient Image: (150-2)

In home patient care occurs in a variety of settings with limited control of ambient lighting. Once the caretaker captures the image 112 on the smartphone, the application 110 normalizes, if the patient image is sufficient for analysis at step 152, a shading of the patient image for comparison with other wound images, such that the shading is based on the ambient lighting. This facilitates comparison and matching with other images by assuring all images 112 for classification appear to have been taken in similar lighting conditions, referred to as shading of the image 112 for effecting lighting correction as part of that smartphone wound imaging system.

Normalizing the shading of the patient image further includes an approach to mitigate the adverse lighting effects based on the so called “Retinex theory.” This approach decomposes an input wound image captured under adverse lighting S into Reflectance R and Lighting I intrinsic images. The effects of the adverse lighting manifest mainly in the lighting image, which is mitigated before being used to construct a final enhanced wound image. By way of background, Retinex theory is a theory of color vision that explains how the brain interprets colors and how it's able to see consistent colors in different lighting conditions. The word “retinex” is a derivation of the words “retina” and “cortex.”

After receiving the patient image 112, normalization of the shading of the patient image occurs by decomposing the patient image into a reflectance component and a lighting component. The lighting component is adjusted by collecting an image set representative of wound images captured from mobile devices of a plurality of vendors, and generating a lighting model 150-2 based on decomposing images of the image set using a wound image of a poor lighting scenario and a wound image of a favorable lighting scenario. This helps to alleviate native differences between the phones and cameras of different vendors. The images of the image set are then decomposed into respective reflectance components and lighting components, such that the lighting components are indicative of structure aware illumination. The application then compares the lighting component of the patient image to the lighting model 150-2 for enhancing the lighting component of the patient image.

The disclosed approach for mitigating the effects of adverse lighting on the semantic segmentation of wound images includes a Deep Retinex Decomposition for Low-light Enhancement method (the Deep Retinex Model or DRM). We utilize the DRM to separate the wound image captured in adverse light into its constituent reflectance and illumination maps, enhance the illumination map before reconstructing an enhanced wound image that appears as it would, if captured in desired lighting. The enhanced wound images are then segmented using U-Net and the change in wound and skin segmentation Dice scores are recorded.

Training the lighting model further includes evaluating a segmentation by labeling, in each image, whether each pixel corresponds to wound, skin or background, and computing segmentation accuracy based on false positives and false negatives of the labeling of the respective image. U-Net is a Convolutional Neural Network (CNN) for semantic segmentation frequently utilized in biomedical applications. Configurations described herein utilize U-Net for segmentation in its wound assessment architecture. Wound segmentation was performed using two distinct U-Net models: 1) Model 1 classified each image pixel as either skin versus non-skin (background) pixels and 2) Model 2 classified image pixels as wound vs non-wound pixels. The masks generated by these two models are then combined to generate a final segmentation output in which each image pixel is labeled as wound, skin or background.

Segmentation accuracy was measured using a Dice Score metric separately for both wound and skin defined as:

$$DiceScore = \frac{2 TP}{2 TP + FP + FN}$$

where TP, FP and FN refer to True Positives, False Positives and False Negatives respectively. Each network was trained with a loss function that was a weighted sum of the binary cross-entropy loss and the Dice score:

$$Loss = BCE - k log DiceScore \\ BCE = - {\sum }_{i = 1}^{N} {g}_{i} log {p}_{i}$$

where pᵢ is the label predicted by the network, gᵢ is the ground truth label gᵢ∈0, 1 and N is the total number of pixels present in the image.

The Deep Retinex Model (DRM) consists of a Decom-Net network, a CNN architecture for image decomposition and Enhance-Net Network, an encoder-decoder network for illumination adjustment. The Decom-Net takes as input a pair of images-a wound image taken in poor lighting and a wound image taken in favorable lighting conditions, and decomposes each image into its respective lighting-independent reflectance and structure-aware smooth illumination map. Each image is concatenated with an initial illumination map created by taking the maximum pixel value across the R, G, and B channels of each pixel. Enhance-Net enhances the illumination map of the poor lighting image. Finally, the final enhanced image is produced by the element-wise multiplication of the reflectance image of the low-light image and the enhanced illumination map produced by Enhance-Net. The DRM takes as input pairs of low-light and normal-light images during training but only a low-light image is input during inference to produce its enhanced version.

The loss function for the Decom-Net consists of three terms: a reconstruction loss L_{recon}, a reflectance consistency loss Lᵢᵣ and an illumination smoothness loss Lᵢₛ with Air and his being the weights to balance the last two loss components:

$$L = {L}_{recon} + {λ}_{ir} {L}_{ir} + {λ}_{is} {L}_{is}$$

As both images in the pair are assumed to share the same reflectance, the input image is reconstructed using its corresponding illumination map and either of the two reflectance maps (original or enhanced). Thus, the reconstruction loss is expressed as follows with λᵢⱼ expressing the weights of the four terms in the loss function:

$${L}_{recon} = {\sum }_{i = low , normal} {\sum }_{j = low , normal} {λ}_{ij} {‖ {R}_{i} \cdot {I}_{j} - {S}_{j} ‖}_{1}$$

The reflectance consistency loss ensures that the respective reflectance of the low-light and normal images are as close as possible, thereby maintaining the color and the visual appeal of the wound image after enhancement.

The illumination smoothness loss ensures that the illumination maps created are smooth while maintaining the object boundaries. It is a modified form of the total variation loss to maintain structure-awareness and involves multiplying the gradients of the illumination and reflectance maps. Consequently, high gradients in the reflectance map that correspond to object boundaries are smoothened less than textures:

$${L}_{is} = {\sum }_{i = low , normal} ‖ ∇ {I}_{i} \cdot exp ( - {λ}_{g} ∇ {R}_{i} ) ‖$$

The loss function used for Enhance-Net consists of a reconstruction loss and an illumination-smoothness loss. The reconstruction loss is calculated between the reconstructed image and the product of the low-light reflectance map and enhanced illumination map. It tries to make their element-wise multiplication close to the input normal light image, which ensures that the illumination map is bright enough to make the enhanced image as close as possible to the input reference image:

$${L}_{recon} = {‖ {R}_{low} ∘ \hat{I} - {S}_{normal} ‖}_{1}$$

The illumination smoothness loss is similar to Decom-Net but here only the reflectance of the low-light image and the enhanced illumination maps are considered:

$${L}_{is} = ‖ ∇ \hat{I} ∘ exp ( - {λ}_{g} ∇ {R}_{low} ) ‖$$

In the utilized color space, the black and white/contrast/lightness is given a separate channel and is independent of the color unlike the RGB color space where it is coupled with all other channels. Thus we can analyze the lightness content of the image independently. To train the DRM, we selected images with mean L-values (LAB space)≤90 from each dataset as the low-light wound images. The DRM tries to make the distribution of input pairs of low light and reference normal light images similar. Hence, for the corresponding reference/normal-light wound image, we chose the image that yielded the highest wound Dice score and a comparable skin Dice score or one that yielded the highest sum of wound and skin Dice scores without any enhancement from each of the datasets.

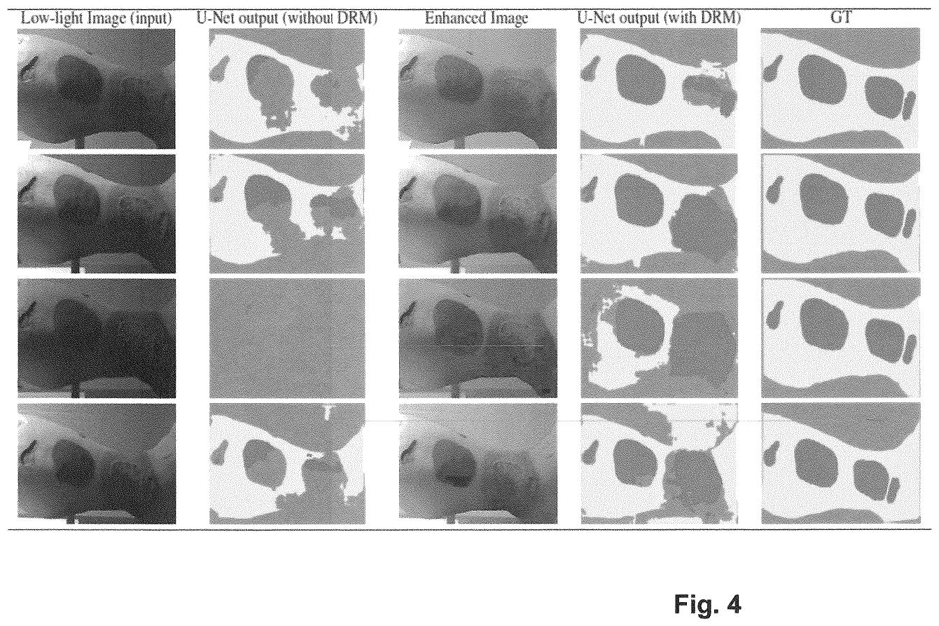

In implementation, the lighting model employs three steps 1) Decomposition of the input image into reflectance and illumination images 2) Enhancement of the low-lighting illumination image, and 3) Reconstruction of an enhanced illumination image by element-wise multiplication of the reflectance and enhanced illumination image. The DRM takes pairs of low-light and normal light images as input during training but requires only single low-light test images during inference. (note that reconstruction here refers to illumination correction, not angle and distance correction performed by the reconstruction model 150-3 described below). FIG. 4 shows results of lighting correction by the lighting model 150-2. Column 1 contains wound images taken in poor lighting. Column 2 shows the U-Net output for the dark image without any enhancement. Column 3 shows dark images after enhancement by our model, while column 4 shows its corresponding U-Net output. Column 5 shows the GT-ground truth mask. The segmentation output is improved significantly after enhancement. Row 3 shows how, for a very dark image, the model 150-2 was unable to create any segmentation mask, but generated a much improved output after enhancement.

Reconstruction of Patient Image for Angle and Distance from Camera: (150-3)

The reconstruction model 150-3 rectifies perspective and scale distortion in the patient image 112, outputting a corrected orthogonal image (one captured along the surface normal at a fixed distance from the wound surface). A small multi-color reference marker is placed next to the wound, and employed to detect the position and size of the marker and a Spatial Transform Network (STN) is used to generate the Inverse Perspective Transformation Matrix (IPTM) that is used to rectify perspective and scale distortion. This allows the patient images 112, which may be captured from a variety of angles and distances depending on the caretaker, to be received and processed as an image taken from a common fixed distance and orthogonal (direct) angle.

The model 150-3 reconstructs the patient image for accommodating variations in the angle and the distance by identifying a perspective distortion in the patient image, identifying a scale distortion in the patient image, and correcting the perspective distortion and the scale distortion for computing the reconstructed image configured for classification with a model of healing progress. The model of healing progress includes at least one of a wound healing model indicative of healing progress and a wound care model indicative of a need for subsequent care of the wound.

A transformation marker physically adhered adjacent the wound provides a normalizing or common reference appearing in the patient image 112. Upon receiving an image including a transformation marker, where the transformation marker results from a manual placement adjacent the wound, the application 110 identifies the perspective distortion and the scale distortion based on analyzing an orientation and size of the transformation marker, computes a reconstruction transform for aligning the transformation marker to an image based on an orthogonal perspective and fixed distance from the wound, and applies the reconstruction transform to the patient image for generating the reconstructed image. In the example configuration, the transformation marker includes a series of concentric circles and a set of control points in an equidistant, circular orientation around the concentric circles, however other suitable arrangement may suffice. For example, a simple concentric array of circles provides an ellipse orientation until perceived orthogonally.

In the reconstruction by the model 150-3, a deep learning approach is invoked to rectify perspective and scale distortion from the wound images. A reference marker of known dimension and color is placed next to the wound during capture and used to calculate rectification parameters. An R-CNN (Convolutional Neural Network) is then used to detect the position and size of the marker in the wound image, a bounding box placed around it and cropped. Finally, a custom Spatial Transformation Network (STN) analyzes the cropped wound image to generate the inverse perspective transformation matrix required to transform the distorted version of the marker to its known, undistorted size. The same global transformation calculated from the marker is also applied to the entire distorted wound image, removing any perspective and scale distortion. The custom STN is trained using a corpus of marker images at different camera orientations and positions.

Perspective projection transforms a 3D object into a 2D image. Different camera positions generate different projected (2D) images of the same 3D object. In smartphone wound imaging, the correct wound area corresponds to the 2D projection when the camera is pointing normal to the wound and is positioned at the focal length (also called the orthogonal image). The wound area in images captured from arbitrary camera positions and angles are affected by artefacts called perspective and scale distortion. Removing perspective distortion in a wound image captured from an arbitrary camera angle requires deriving the Inverse Perspective Transformation Matrix (IPTM). The IPTM is a 3×3 homogenous transform matrix Tₚ that can transform the distorted image into the orthogonal image, nullifying the effect of the arbitrary selection of origin, camera orientation and scale in the coordinate frame of the image.

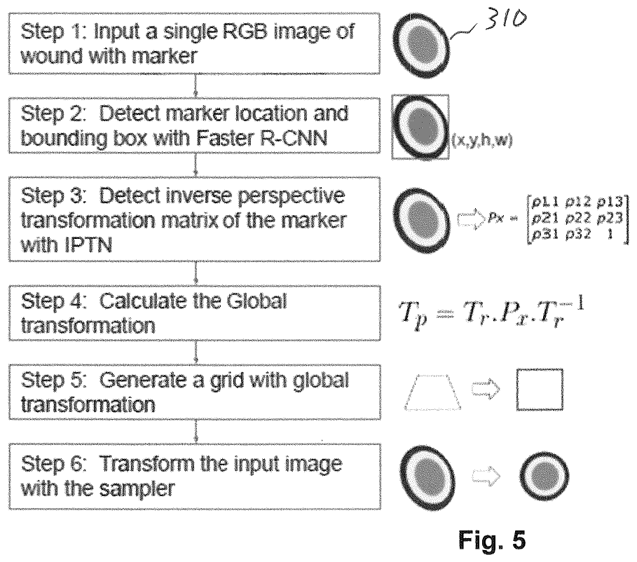

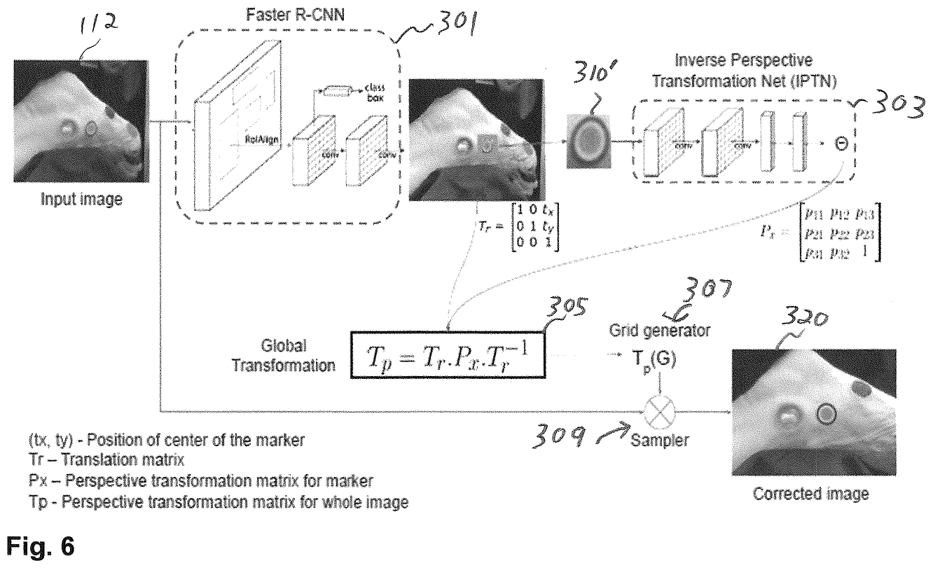

FIG. 5 shows a flowchart of the reconstruction and rectification process, and FIG. 6 shows the transforms in the reconstruction of FIG. 5. Referring to FIGS. 1, 5 and 6, the overall flow of the reconstruction performed by the reconstruction model 150-3 is as follows.
Step 1: An RGB image 302 defined by the patient image 112 is captured with a reference marker 310 placed next to the wound 114.
Step 2: an R-CNN 304 detects the reference marker in the image. From the output, the marker image is cropped and we generate the translation matrix Tᵣ from the center location of the detected marker.
Step 3: Using the cropped marker image, the STN generates the parameters of an IPTM centered at the marker center, which rectifies perspective and scale distortion in the reference marker image.
Step 4: The global IPTM is calculated using the IPTM learned from the marker. It transforms the whole image to be centered at the center of the marker in the input image. This operation uses a 3×3 translation matrix Tᵣ, generated from the center of the detected marker, x_{center} and y_{center}. Finally, the global IPTM to transform the whole image is calculated by combining the IPTM and translation matrix Tᵣ, as shown below:

$${T}_{r} = [ \begin{matrix}1 & 0 & {x}_{center} \\ 0 & 1 & {y}_{center} \\ 0 & 0 & 1\end{matrix} ]$$

$${P}_{x} = [ \begin{matrix}{p}_{11} & {p}_{12} & {p}_{13} \\ {p}_{21} & {p}_{22} & {p}_{23} \\ {p}_{31} & {p}_{32} & 1\end{matrix} ]$$

$${T}_{p} = {T}_{r} . {P}_{x} . {T}_{r}^{- 1}$$

Step 5: The global transform generated is applied to generate the grid mapping from input to output image pixels.
Step 6: Using the mapping, the sampler samples the input image to produce an output image free from perspective and scale distortion.

The disclosed configuration employs transfer learning and pre-trains the R-CNN model with images. Transfer learning improved the R-CNN model's accuracy and generalizability, and reduced overfitting. We captured 17,000 images of a phantom wound with a marker placed on the leg and foot moulages (synthetic body parts) with wounds. The approach annotates the marker in the images by utilizing the marker color information that is manually verified before training the model. To further improve the accuracy of the R-CNN model 301 and increase its robustness to diverse lighting, capture scenarios and camera configurations, color, brightness, contrast and adding Gaussian noise augmentations were applied to each input image. The location of the marker detected by the marker detection step is used to generate a cropped marker image and also to derive a translation matrix (Tᵣ) with marker center as translation parameters.

The Inverse Perspective Transformation Network (IPTN) 303 uses the cropped marker image 310′ to generate the IPTM that rectifies the perspective and scale distortion of the cropped marker image. The IPTM matrix has nine elements and eight Degrees Of Freedom (DOF). It is scaled such that p₃₃=1, yielding the orthogonal image based on the cropped marker image's origin. The cropped marker image is processed rather than the entire image because in the captured image, the marker is the only known object in the image. image. All other objects in the image are unknown. Prior knowledge of the standard size of marker enables the inverse transformation to be calculated and the orthogonal image to be generated. We trained a deep STN network to produce the IPTM. We adopt the network notation where conv[N,w,s,p] denotes a convolution layer with N filters of size w×w, with stride s and p pixel padding, ƒ_{C}[N] is a fully connected layer with N units, and max[s] is an s×s max-pooling operation with stride s. All layers use Rectified Linear (ReLU) non-linearity and linear outputs from the final fully connected layer. The network structure is: conv[16, 3, 1, 1]-conv[16, 3, 1, 1]-conv[16, 3, 1, 1]-max[2]-conv[16, 3, 1, 1]-conv[16, 3, 1, 1]-conv[16, 3, 1, 1]-max[2]-conv[16, 3, 1, 1]-conv[16, 3, 1, 1]-conv[16, 3, 1, 1]-max[2]-fc [512]-fc [512]-fc [8]. The deep network outputs 8 values that correspond to the 8 elements of the IPTM, Pₓ. The 9^{th} element of the IPTM is always set to 1.

The IPTN was trained on a dataset generated using a simulator that has advanced 3D graphics, sensor and noise, facilitating an accurate, dynamic, simulated environment. In the simulator, we placed an image of the marker next to a phantom wound and placed the camera in different poses (orientation and position) to capture the image and corresponding IPTM as ground truth. The ground truth vector consists of the element of the scaled IPTM matrix (p₃₃=1), which is computed in the simulator. The ground truth is a vector of size 1×8, which consist of 8 elements of the IPTM Pₓ. In the simulator, we generated a dataset of images each with corresponding ground truth, by changing the pose of the camera. A 90%: 10% train: test split was created. The network was trained using the RMSProp optimizer using a Root Mean Squared Error (RMSE) loss function:

$$RMS E = \sqrt{\frac{1}{n} {\sum }_{i = 1}^{n} {( {Y}_{i} - {\hat{Y}}_{ι} )}^{2}}$$

A global transformation step 305 generates the IPTM Tp for the whole image (Equation 8) by combining the IPTM Pₓ previously generated for the marker with the translation matrix Tr.

A grid generator 307 generates a grid map, which maps all the pixels in the input image to the output image based on the IPTM Tₚ calculated in the previous step. Not all the pixels in the output image have a valid mapping from the input image using the IPTM, resulting in missing pixels. A sampler step 309 fills in these missing pixels using information contained in the nearby pixels. The sampler uses a grid to sample the input image pixels and a Bi-linear transformation similar to a hierarchical spatial transformer network. Each output pixel is calculated using the four nearest pixels. The output pixel's value is weighted by its distance from the closest pixel in the grid as expressed

$$O ( p ) = {\sum }_{n = 1}^{4} I ( {p}_{n}^{′} ) \vert ( 1 , 1 ) - \vert {p}_{n}^{′} - p {‖}_{2}^{2}$$

Where O is the output image 320, I is the input image, p and p′ₙ are integer coordinates on the image, p′ₙ is a 4-pixel neighbor (top-left, top-right, bottom-left, bottom-right) of p.

### Scoring of Wound Healing: (150-4)

Once the patient image 112 has been augmented for brightness and lighting, and reconstructed for orientation and distance, a scoring approach is employed to provide an objective assessment of healing that avoids subjective deviations that can vary from clinician or caretaker. Control returns to the AI-driven smartphone app 110 to analyze wound images and score wounds based on a clinically validated wound healing rubric, thus addressing a need for facilitate fast, accurate wound healing assessment in patients' homes, remote from a hospital or medical facility.

Configurations herein generate the wound score by accessing a database of wound images, and identifying a plurality of attributes, each of the attributes indicative of a healing state of the wound under care. The application 110 or 120 compares the attributes of the wound under care to the attributes of the wound images in the database, and generates, for each of the attributes, an evaluation score for each of the attributes of the wound under care based on the comparison, and then computes the wound score based on each of the evaluation scores. Assessment is based on a Photographic Wound Assessment Tool (PWAT), a clinically validated wound grading rubric. The PWAT evaluates eight attributes of wounds from an image: 1) Size 2) Depth 3) Necrotic Tissue Type 4) Necrotic Tissue Amount 5) Granulation Tissue Type 6) Granulation Tissue Amount 7) Edges and 8) Skin viability. Each PWAT sub-score grades a single wound attribute with a score of 0 (best), 1, 2, 3 or 4 (worst) and higher scores indicate a worse wound condition. All 8 PWAT sub-scores are summed to generate a total PWAT wound score in an integer range of 0-32.

The wound healing model 150-4 was trained on a corpus of wound images to define attributes and/or labels, where the attributes include at least one text attribute based on a numeric value corresponding to the respective image, and at least one image attribute based on the wound under care. A modest amount of image correction was performed as preprocessing.

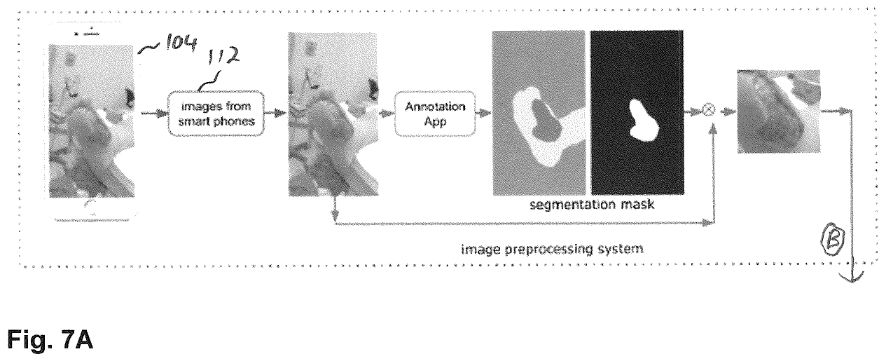

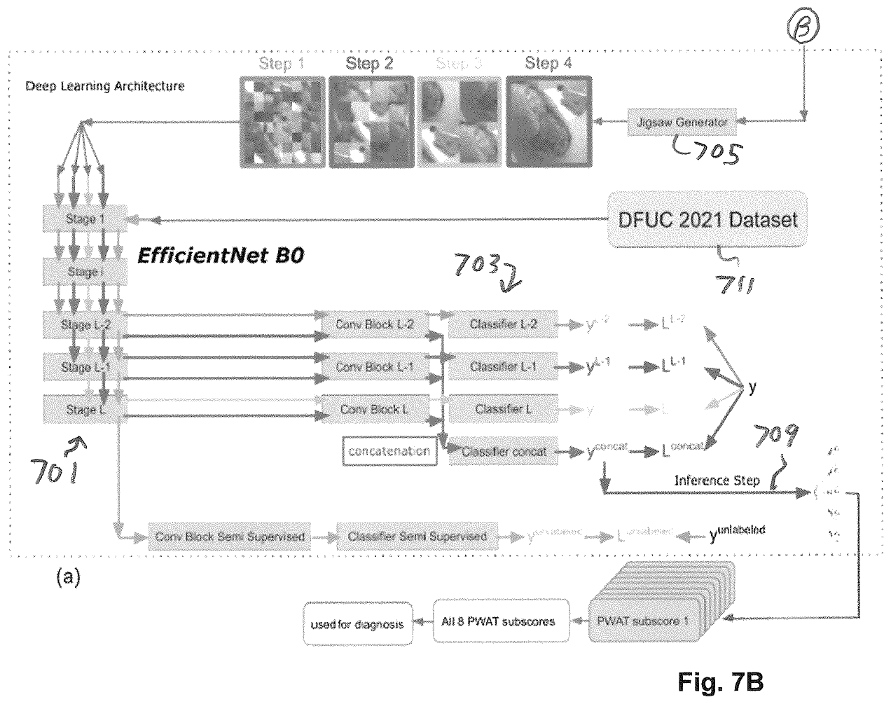

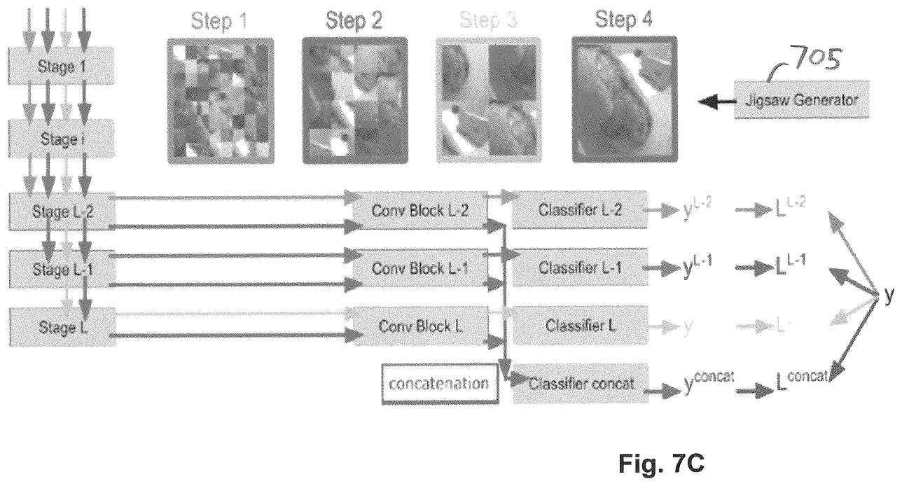

FIGS. 7A-7B show the learning architecture for accessing 8 PWAT sub-scores for the wound healing model 150-4. This architecture is composed of 3 main components: a semi-supervised learning component, the PMG (Progressive Multi-Granularity) component and the baseline deep learning model: EfficientNet B0. FIG. 7C shows the deployment or production use of the wound healing model.

The PMG (Progressive Multi-Granularity) mechanism can be implemented as a feature extractor with any suitable image analysis model. Suppose F is the feature extractor with L stages. Its intermediate stages have output feature-map: F^{l}∈R^{Hl×Wl×Cl}. Here Hₗ, Wₗ, Cₗ are the height, width and number of channels of the feature map at l-th stage, l=1, 2, . . . , L. The next step is to calculate the classification loss on the feature-map from different intermediate stages. The new convolution block H_{conv}^{l} takes l-th intermediate stage output, F^{l}, as input. Its output was reduced to a vector representation:

$${V}^{l} = {H}_{conv}^{l} ( {F}^{l} ) \vert$$

Then, a classification module H_{class}^{l} with two fully-connected stages, calculates the probability distribution for each classe for the l-th stage:

$${y}^{l} = {H}_{class}^{l} ( {V}^{l} )$$

After calculating the last S stages 701: l=L, L−1, . . . , L−S+1, the outputs from them are concatenated as:

$${V}^{concat} = concat [ {V}^{L - S + 1} , … , {V}^{L - 1} , {V}^{L} ]$$

It is then input into a classifier 703:

$${y}^{concat} = {H}_{class}^{concat} ( {V}^{concat} )$$

In traditional CNN models, training the entire network directly in traditional CNN models means learning all the granularities simultaneously. In progressive training, the low stage is trained first and then new stages are added for training progressively. The PMG mechanism allows the network to first exploit discriminative information from local details such as textures because the low stage has a limited receptive field and representation ability. When the features are gradually input into higher stages, the model can locate discriminative information from local details to global structures. The outputs from each stage and the output from the concatenated features are input into the cross entropy (CE) L_{CE}. The loss between ground truth label y and prediction probability distribution is calculated

$${L}_{CE} ( {y}^{l} , y ) = - {\sum }_{i = 1}^{m} {y}_{i}^{l} \times log ( {y}_{i}^{l} )$$

$$and$$

$${L}_{CE} ( {y}^{concat} , y ) = - {\sum }_{i = 1}^{m} {y}_{i}^{concat} \times log ( {y}_{i}^{concat} )$$

In each training iteration, the data d will be used for S+1 times but only to obtain the output for each stage in each time. All parameters used in each stage are updated even though they may already be updated in the previous stages, which helps all stages in the model work together.

The notion of a jigsaw puzzle is used here to generate input images for different stages of progressive training. The jigsaw puzzle generator 705 generates different granularity regions so that the model can learn the corresponding granularity level's information which is specific at each training step. The input image d∈R^{3×W×H} is equally split into n×n patches with 3×W/n×H/n dimensions. The patches are shuffled randomly and merged together into a new image P(d, n) so that the hyper-parameter n controls the patches' granularities.

The correct hyper-parameter n for each stage should guarantee that the patches' size should be smaller than the receptive field at the corresponding stage and the patches' size should increase proportionately as the receptive fields of the stages increase. For the l-th stage, n is chosen as:

$$n = {2}^{L - l + 1}$$

During training, as shown in FIG. 7C, the jigsaw puzzle generator 705 augments training data batch d to generate several augmented batches P(d, n), which all have the same label y. The batch P(d, n) with n=2^{L-l+1} is input to the l-th stage which generates the output y^{l}, then all the parameters used in this process will be updated in this propagation. All the jigsaw generator augmented data batches are input sequentially into the network by S+1 steps.

During inference at 709, the original images are input into the trained model without the jigsaw puzzle generator. To only utilize y^{concat} for prediction, the FC layers for the other three stages are removed and the final result C₁:

$${C}_{1} = arg max ( {y}^{concat} )$$

The prediction from each stage has unique and complementary information from a specific granularity. To obtain a better performance, all outputs are combined together with equal weights and the multi-output combined prediction C₂ is:

$${C}_{2} = arg max ( {\sum }_{i = L - S + 1}^{L} {y}^{l} + {y}^{concat} )$$

Thus, in deployment and classification as in FIGS. 7A-7B, comparing the patient image 112 further includes identifying a plurality of features descriptive of the wound under care based on the patient image 112, and comparing the wound under care to the wound healing model 150-4. Using the model 150-4, the application retrieves, based on the comparison, an integer score for each feature of the plurality of features; and computes the wound score based on an aggregation of the integer scores to compute the PWAT score.

Training the wound healing model 150-4, as shown in FIG. 7C, further includes receiving a training corpus of a plurality of images of healing wounds, and annotating, for each image of healing wounds, an integer score for each feature of the plurality of features. The jigsaw puzzle generator 705 is for identifying and learning different granularity regions in each respective image in the training corpus.

A semi-supervised learning approach applied for the wound healing model 150-4 was inspired mainly from a rotation degree Self-Supervised Learning. This includes a simple but effective algorithm for semi-supervised image classification via self-supervision. The dataset for the semi-supervised learning method consists of pairs of images and labels (x, y)∈SL and unlabeled images x∈S_{U}. Usually S_{L} and S_{U} are sampled from the same distribution p(x) and S_{L} is S_{U}′ subset with labels. However, S_{L} is a WoundNet dataset and S_{U} is the DFUC dataset 711 in our case, as mentioned in Sub-section II-A. It is possible to sample S_{L} from p(x) but sample S_{U} from q(x), a different yet related distribution [56]. This semi-supervised learning method trains a prediction function ƒ_{θ}(x) with parameter θ on a combination of S_{L} and S_{U} to obtain better model performance than training on S_{L} alone. During the training process, two batches of data are sampled from the labeled dataset S_{L} and unlabeled dataset S_{U} separately in each step:

s_{L}=b(xᵢ∈S_{L})

s_{U}=b(xⱼ∈S_{U})

Then they are input into the shared baseline model ƒ_{θ}(x), which is EfficientNet B0 in our case. The labeled batch s_{L} and the unlabeled batch s_{U} are input into ƒ_{θ}(x) so that its softmax layer generates prediction vectors from them respectively:

zᵢ=ƒ_{θ}(s_{L})

zⱼ=ƒ_{θ}(s_{U})

The ground truth labels yᵢ are used for computing the supervised cross-entropy loss L_{labeled}(yᵢ, zᵢ). The DFUC 2021 dataset's label is considered as the dataset S_{U}'s label, which is used as the proxy labels yⱼ to compute the cross-entropy loss L_{unlabeled}(yⱼ, zⱼ) for the unsupervised cross-entropy loss:

$$\begin{matrix}{L}_{labeled} ( {y}_{i} , {z}_{i} ) = - \frac{1}{\vert {s}_{L} \vert} {\sum }_{i ∈ {s}_{L}} {\sum }_{k ∈ K} {y}_{ik} log ( {z}_{ik} ) \\ {L}_{unlabeled} ( {y}_{j} , {z}_{j} ) = - \frac{1}{\vert {s}_{U} \vert} {\sum }_{j ∈ {s}_{U}} {\sum }_{t ∈ T} {y}_{jt} log ( {z}_{jt} )\end{matrix}$$

The final loss function is defined as the weighted sum of the supervised cross-entropy loss and the unsupervised cross-entropy loss:

$${L}_{final} = {L}_{labeled} ( {y}_{i} , {z}_{i} ) + ω {L}_{unlabeled} ( {y}_{j} , {z}_{j} )$$

The parameter θ will be updated in backpropagation after minimizing the final loss function L_{final}. The unsupervised cross-entropy loss L_{unlabeled} (yⱼ, zⱼ) can be considered as a regularization term in the final loss function and ω>0 is a regularization hyperparameter that controls the relative contribution of unsupervised learning in the semi-supervised learning process.

### Subsequent Care Recommendation: (150-5)

The caretaker 102 determines a recommendation for continued care before leaving the patient 106. As with wound healing computed by 150-4, the wound care model 150-5 computes whether additional care is needed by accessing a database of decision support evaluations, where the decision support evaluations each include an evaluated wound image and a narrative segment. The narrative segment is indicative of a state of healing of the wound under care, and a result concludes a need for the additional care based on comparing the wound under care to, for a plurality of the decision support evaluations, the evaluated wound image and the narrative segment corresponding to evaluated wound image. The need for additional care includes at least one of continuing current care, referring for additional care, and referring for urgent care. The narrative description associated with the respective learned images, once trained into the wound care model 150-5, is used to determine which wounds are healing sufficiently to allow home care to continue, and which need to be referred, possibly urgently, to an increased level of care.

The wound care model 150-5 is based on a database of decision support evaluations, developed from training a wound care model for bimodal classification based on text attributes and visual attributes from a training dataset, the training dataset including images of wounds and a corresponding treatment conclusion indicative of a need for additional care.

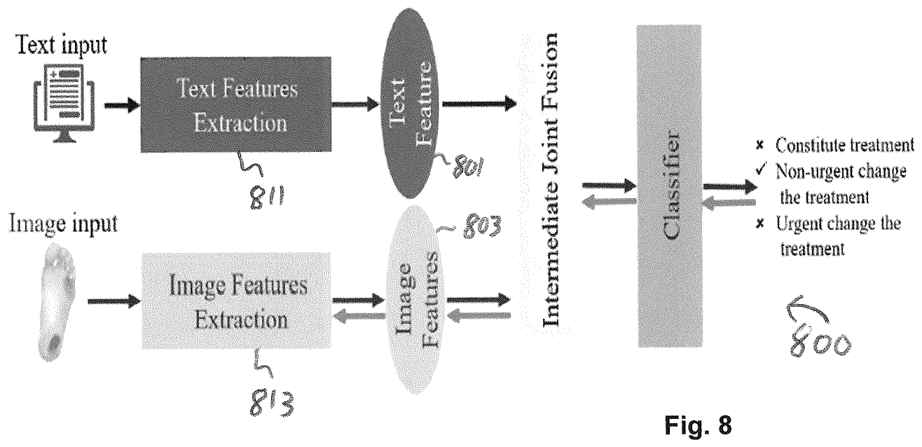

FIG. 8 shows a Multimodal Classifier Pipeline 800 implementing a fusion of expertise from Natural Language Processing (NLP) and Convolutional Neural Networks (CNN) for robust decision-making. EHR input 134 undergoes NLP-driven feature extraction for text feature extraction 811, capturing textual insights. An intermediate joint fusion algorithm is used for combining text 801 and image 803 features. Concurrently, image input is processed through a transformer-enhanced CNN model, capturing intricate visual features for image feature extraction 813. This comprehensive approach integrates extracted text and image features into a classifier, enabling accurate classification into three distinct decisions: 1) maintaining current treatment, 2) See wound expert (non-urgent), and 3) See wound expert (urgently) as the decision support output.

Multimodal fusion methods include early fusion, intermediate fusion, and late fusion. Early fusion merges features from different modalities at the input level, while late fusion combines decisions from separate models. Intermediate fusion, as employed herein, involves extracting high-level features independently from each modality (wound images and clinical notes) before combining them into a unified representation for final classification.

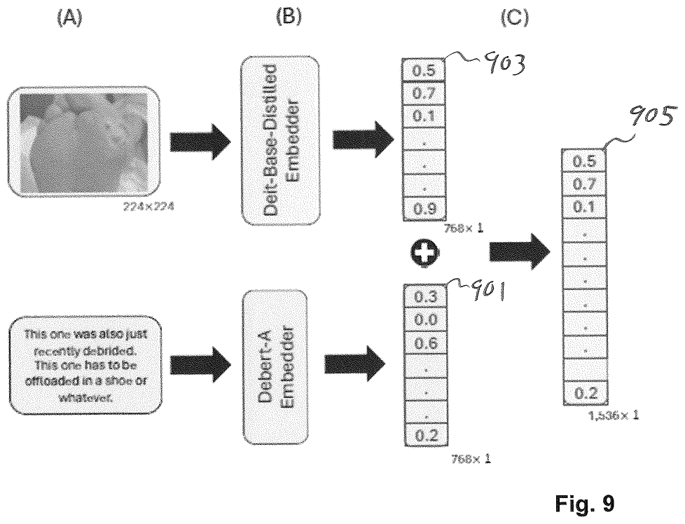

FIG. 9 shows intermediate fusion of multimodal features as in FIG. 8. In the illustration of the intermediate fusion process of FIG. 9, col. (A) depicts Input modalities (image and text), col. (B) shows modality-specific embedders (DeiT-Base-Distilled for images, DeBERTa-base for text), resulting in col. (C): extracted embedding vectors concatenated into a 1,536-dimensional combined representation. This approach effectively retains modality-specific characteristics while enabling their integration, facilitating a richer and more nuanced feature space. Therefore, the multimodal approach includes, for each of a plurality of entries in the training dataset, generating a text vector 901 based on the text attributes, and an image vector 903 based on the visual attributes. The text vector and the image vector are then concatenated to form a multimodal vector 905.

Following training, deployment or classification includes comparing the patient image to the wound care model by extracting visual features of the patient image 112, and matching the extracted visual features to the wound care model 150-5. The recommendation is then generated the based on text attributes of the entries having a correspondence of the visual features of the patient image 112.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of gathering and classifying wound images, comprising:
receiving a patient image from a personal device, the patient image containing an image of a wound under care, the image of the wound under care gathered with an ambient lighting, angle and distance of the personal device relative to the wound under care;
evaluating a sufficiency of a patient image for analysis, the patient image depicting a wound under care;
normalizing, if the patient image is sufficient for analysis, a shading of the patient image for comparison with other wound images, the shading based on the ambient lighting;
reconstructing the patient image for accommodating variations in the angle and the distance; and
analyzing the reconstructed image for rendering a recommendation including the wound score and the additional care.

2. The method of claim 1, further comprising:
if the patient image is insufficient for analysis, discarding the patient image; and
rendering an indication, via the personal device, that the image is insufficient for analysis and indicating a need for a subsequent image gathering.

3. The method of claim 2 wherein evaluating the sufficiency of the patient image further comprises:
computing an index for each of brightness, compression, and blurriness of the patient image; and
computing an image assessment based on a comparison of the computed indices; and
rejecting the patient image if the image assessment does not meet a sufficiency threshold.

4. The method of claim 3 further comprising:
training a model based on stored images of previously classified wounds;
comparing the patient image to the stored images of the model; and
computing the indices of brightness, compression, and blurriness based on the comparison.

5. The method of claim 3 102, further comprising normalizing the shading of the patient image by:
decomposing the patient image into a reflectance component and a lighting component; and
adjusting the lighting component.

6. The method of claim 5, further comprising adjusting the lighting component by:
collecting an image set representative of wound images captured from mobile devices of a plurality of vendors; and
generating a lighting model based on decomposing images of the image set using a wound image of a poor lighting scenario and a wound image of a favorable lighting scenario;
decomposing the images of the image set into respective reflectance components and lighting components, the lighting components indicative of structure aware illumination; and
comparing the lighting component of the patient image to the lighting model for enhancing the lighting component of the patient image.

7. The method of claim 6, wherein training the lighting model further comprises:
evaluating a segmentation by labeling, in each image, whether each pixel corresponds to wound, skin or background, and
computing segmentation accuracy based on false positives and false negatives of the labeling of the respective image.

8. The method of claim 1, wherein reconstructing the patient image further comprises:
identifying a perspective distortion in the patient image;
identifying a scale distortion in the patient image;
correcting the perspective distortion and the scale distortion for computing the reconstructed image configured for classification with a model of healing progress.

9. The method of claim 8 wherein the model of healing progress includes at least one of a wound healing model indicative of healing progress and a wound care model indicative of a need for subsequent care of the wound.

10. The method of claim 8 further comprising:
receiving an image including a transformation marker, the transformation marker resulting from a manual placement adjacent the wound;
identifying the perspective distortion and the scale distortion based on analyzing an orientation and size of the transformation marker;
computing a reconstruction transform for aligning the transformation marker to an image based on an orthogonal perspective and fixed distance from the wound; and
applying the reconstruction transform to the patient image for generating the reconstructed image.

11. The method of claim 10 wherein the transformation marker includes a series of concentric circles and a set of control points in an equidistant, circular orientation around the concentric circles.

12. The method of claim 1, further comprising:
generating a wound score based on the reconstructed image, the score indicative of a healing progress of the wound under care;
computing, based on a comparison of images of other wounds with the reconstructed image, whether additional care is needed for the wound under care; and
rendering a recommendation including the wound score and the additional care.

13. A system for gathering and classifying wound images by onsite, remote caretakers, comprising:
a personal device, the personal device configured for taking a patient image containing an image of a wound under care, the image of the wound under care gathered with an ambient lighting, angle and distance of the personal device relative to the wound under care;
a smartphone app configured for evaluating a sufficiency of a patient image for analysis, the patient image depicting a wound under care; and
a wireless link coupling the personal device to a server,
the smartphone app configured to engage the server via the wireless link for:
normalizing, if the patient image is sufficient for analysis, a shading of the patient image for comparison with other wound images, the shading based on the ambient lighting;
reconstructing the patient image for accommodating variations in the angle and the distance; and
analyzing the reconstructed image for rendering a recommendation including the wound score and the additional care.

14. A computer program embodying program code on a non-transitory computer readable medium that, when executed by a processor, performs steps for implementing a method for gathering and classifying wound images, the method comprising:
receiving a patient image from a personal device, the patient image containing an image of a wound under care, the image of the wound under care gathered with an ambient lighting, angle and distance of the personal device relative to the wound under care;
evaluating a sufficiency of a patient image for analysis, the patient image depicting a wound under care;
normalizing, if the patient image is sufficient for analysis, a shading of the patient image for comparison with other wound images, the shading based on the ambient lighting;
reconstructing the patient image for accommodating variations in the angle and the distance;
generating a wound score based on the reconstructed image, the score indicative of a healing progress of the wound under care; and
analyzing the reconstructed image for rendering a recommendation including the wound score and the additional care.
